(19) United States
(12) Patent Application Publication
(10) Pub. No.: US 2024/0008912 A1
(43) Pub. Date: Jan. 11, 2024

(54) ELECTROSURGICAL ENERGY HARVESTING

(71) Applicant: Medtronic, Inc., Minneapolis, MN (US)

(72) Inventor: Mohammad MIRI, Longmont, CO (US)

(21) Appl. No.: 18/338,696

(22) Filed: Jun. 21, 2023

Related U.S. Application Data
(60) Provisional application No. 63/358,382, filed on Jul. 5, 2022.

(51) Int. Cl. A61B 18/14

(52) U.S. Cl. CPC A61B 18/14; A61B 2018/00702

(57) ABSTRACT

An electrosurgical device configured to harvest RF energy to provide power to one or more loads. The electrosurgical device including a distal portion having two electrodes configured to introduce electrical current into tissue and a proximal portion coupled to an electrical connector. The electrical connector is configured to provide a treatment signal and a continuous signal to an energy harvesting assembly housed within the electrosurgical device. The energy harvesting assembly includes a transformer configured to isolate and reduce the treatment signal to a lower voltage, an AC-DC converter configured to convert the AC signal to DC, and a DC-DC regulator configured to output a fixed voltage. The one or more loads can be electrically coupled to the energy harvesting assembly such that the one or more loads are powered by the fixed voltage.

### CROSS-REFERENCE TO RELATED APPLICATIONS

This application claims the benefit of U.S. Provisional Application Serial No. 63/358,382, filed Jul. 5, 2022, the disclosure of which is incorporated by reference herein.

### FIELD

Embodiments of the present disclosure relate generally to the field of electrosurgical energy delivery, and more particularly to harvesting an available treatment signal in electrosurgery to power electrosurgical handpiece loads.

### BACKGROUND

Electrosurgical devices for applying electrical energy to tissue are commonly used in surgical procedures for hemostatic sealing and coagulation of soft tissue and bone at the operative site. Such electrosurgical devices can be used for, but not limited to orthopedic, spine, thoracic, and open abdominal surgery.

An electrosurgical device may comprise a hand piece having a distally mounted end comprising one or more electrodes. The one or more electrodes can be positioned against the tissue such that electrical current is introduced into the tissue. The generated heat can be used to cut, coagulate or induce metabolic processes in the target tissue. An electrosurgical generator generally provides power and electrical energy in the form of radio frequency (“RF”) energy to one of two handpiece topologies, the monopolar and the bipolar.

During monopolar operation, current is introduced into the tissue by an active electrode and returned through a return electrode separately located on a patient's body. Therefore, the monopolar handpiece has only one wire for the treatment signal in the monopolar connector and the second contact known as the return signal exists in a different connector known as return pad connector.

During bipolar operation, current is introduced into and returned from the tissue by active and return electrodes. The bipolar handpiece therefore provides both electrodes required for the treatment to the bipolar connector. To achieve this, bipolar handpieces usually use a 3-pin connector capable of providing a high-power treatment signal and a continuous low power signal.

Conventional electrosurgical devices used for electrosurgical tissue treatment face an array of challenges that can vary across procedures. Some challenges that arise are inconsistent illumination of the treatment area and inability to verify the electrosurgical device is compatible with a certain electrosurgical generator. Traditional solutions to address these issues typically require additional wiring or the inclusion of a battery within the electrosurgical device. These approaches increase costs and can require the purchase of new equipment.

Accordingly, improved systems and methods are desired for enhancing the capabilities of electrosurgical devices without changing the generator or requiring a second electrical connection to the electrosurgical device.

### SUMMARY

The techniques of this disclosure generally relate to an electrosurgical device configured to enable energy extraction from both detection and treatment signals to power accessories, so as to increase functionality of the electrosurgical device without requiring any change to wiring or an electrosurgical generator.

In one aspect, the present disclosure provides an electrosurgical device configured to harvest RF energy to power to one or more loads. The electrosurgical device can include a distal portion and a proximal portion. The distal portion can include two electrodes configured to introduce electrical current into tissue. The proximal portion can be coupled to an electrical connector configured to provide a treatment signal and a continuous signal to an energy harvesting assembly housed within the electrosurgical device. The energy harvesting assembly can include a transformer configured to reduce the treatment signal to a lower voltage, an AC-DC converter configured to convert the AC signal to DC, and a DC-DC regulator configured to output a fixed voltage. The one or more loads can be electrically coupled to the energy harvesting assembly such that the one or more loads are powered by the fixed voltage.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Subject matter hereof may be more completely understood in consideration of the following detailed description of various embodiments in connection with the accompanying figures, in which:

FIG. 1 is a schematic view depicting an electrosurgical device, in accordance with the prior art.

FIG. 2 is a schematic view depicting an electrosurgical device, according to an embodiment.

FIG. 3 is a flow chart of a method for powering a load of an electrosurgical device, according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

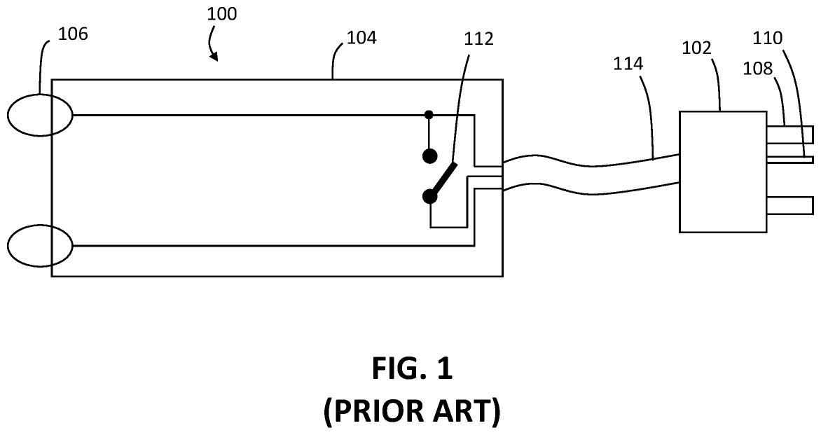

FIG. 1 is a partial schematic diagram of an electrosurgical device 100 configured to transmit RF energy at a treatment site to provide hemostatic sealing and coagulation of soft tissue and bone. Electrosurgical device 100 includes connector 102 and bipolar handpiece 104 having two electrodes 106.

Connector 102 includes large pins 108 and small pin 110 and is configured to be in electrical communication with an electrosurgical generator (not pictured) and a proximal end of bipolar handpiece 104 such that power signals are delivered to electrodes 106 at a distal end of bipolar handpiece 104. In embodiments, connector 102 can be a 3-pin connector capable of providing a high-power treatment signal and a low-power continuous signal via cable 114. Large pins 108 are for a high-power treatment signal and small pin 110 uses a low-power continuous signal to detect a button press on the handpiece. When the button is pressed, the circuit is closed by switch 112 and the treatment signal is provided. In embodiments, an example of the treatment signal is at 469 KHz and 20 W to 220 W while an example of continuous signal is 47 KHz.

It will be appreciated that the terms “proximal” and “distal” are used herein with reference to a clinician gripping a handpiece. Thus, electrodes 106 are distal with respect to the more proximal handle or gripping portion of bipolar handpiece 104. However, surgical devices are used in many orientations and positions, and these terms are not intended to be limiting and absolute.

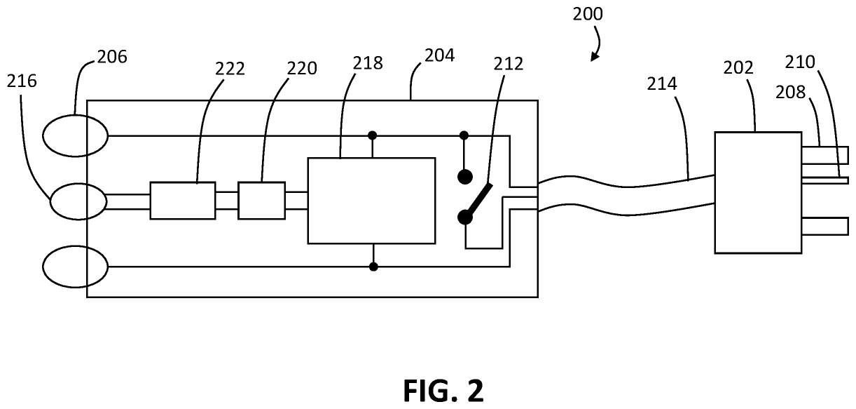

FIG. 2 depicts a schematic diagram of an electrosurgical device 200 configured to convert a small portion of the RF signal used for treating the tissue or for detecting a button press to DC voltage. The DC voltage can be used to power loads 216 of the electrosurgical device such as one or more LEDs, a microprocessor, and a timer.

Electrosurgical device 200 includes connector 202 and bipolar handpiece 204 having two electrodes 206. Connector 202 includes large pins 208 and small pin 210 and is configured to be compatible with existing electrosurgical power source or bipolar energy supplies. For example, the transcollation sealing energy supplied by the Aquamantys® System (available from Medtronic Advanced Energy of Portsmouth, N.H.) may be used. U.S. Pat. Nos. 6,558,385; 6,702,810, 6,953,461; 7,115,139, 7,311,708; 7,537,595; 7,645,277 and 7,811,282 also describe bipolar ablation energy systems suitable for use with embodiments of the present disclosure.

Accordingly, electrosurgical device 200 connects to a source of electrical energy via connector 202. Because connector 202 is designed for compatibility with preexisting generators, the prongs are standardized based on the desired generator. In embodiments, more or less large prongs and short prongs may be used depending on the requirements of the generator. As such, powering loads 216 without a change in connector 202 or generator hardware enables additional functionality to be included within electrosurgical device 200 while maintaining compatibility with existing generators. Additionally, the present disclosure minimizes the wiring coming from electrosurgical device 200 so as to not be cumbersome in operation and storage. Selectively providing electrical energy to electrosurgical device 200 may be accomplished via an actuator on the handle at the proximal end of handpiece 204. Switch 212 is associated with the actuator such that a treatment signal is provided to electrodes 206 upon actuation. Electrical pathways within bipolar handpiece 206 can be formed as conductive arms, wires, traces, other conductive elements, and other electrical pathways formed from electrically conductive material such as metal and may comprise stainless steel, titanium, gold, silver, platinum or any other suitable material.

One aspect of the present disclosure is the inclusion of an energy harvesting assembly within electrosurgical device 200 that is configured to harvest energy from the electric signals provided by an electrical power supply to power one or more loads 216 without affecting operation of electrodes 206.

The energy harvesting assembly can include transformer 218, AC-DC converter, 220, and DC-DC regulator 222. In embodiments, the signal path can start from the button detect signals. Transformer 218 serves to bring down the high voltage treatment signal to a lower range acceptable for the DC circuitry and at the same time isolate the DC and AC circuits. AC-DC converter 220 can include any known means of converting AC signal to DC such as by a diode bridge and capacitor. DC-DC regulator 222 regulates the output voltage of the energy harvesting assembly to a fixed value acceptable for one or more loads 216. DC/DC regulator 222 is used due to sensitivity to voltage level and difference in power level when the electrosurgical device is in operation as touching tissue, bone, or saline can result in wide variations in the treatment signal. The energy harvesting assembly can accordingly reduce the high voltage RF treatment signal to a lower voltage without requiring a battery or additional DC connection, such as a USB.

In embodiments, the effect of the load on the treatment signal can be adjusted by the amount of current being used. For example, a typical minimum power for an electrosurgical device, such as electrosurgical device 200, is 20 W and an load, such as an LED can use less than 0.1 W, staying well within an acceptable tolerance of the electrosurgical device (e.g. +/−4 W at 20 W nominal power).

Embodiments of the present disclosure are operable with various form factors of bipolar handpiece 204. In embodiments, bipolar handpiece 204 can include a distal shaft separating electrodes 206 from the handle. The distal shaft can comprise various materials and shapes such that the distal shaft is rigid, semi rigid, or flexible and the distal shaft can be angled, straight, or bendable. Regardless, the distal shaft can be sized and configured for the specific procedure or targeted area intended. In some embodiments, the distal shaft can be telescoping or retractable. In addition, the distal shaft can comprise a unitary structure or may comprise separately formed members which are permanently or removably joined. The distal shaft may be separable from bipolar handpiece 204 in embodiments where the distal shaft and electrodes 206 comprise a disposable portion of electrosurgical device 200.

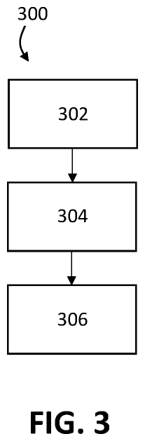

Referring now to FIG. 3, a method 300 for powering an load of an electrosurgical device using an RF signal is depicted according to an embodiment.

At 302, the high voltage treatment signal is brought to a lower range that is acceptable for DC circuitry and the DC and AC circuits are isolated. In embodiments, this can be accomplished by the addition of a transformer.

At 304, the AC signal is converted to DC by using known circuits, such as a diode bridge and capacitor.

At 306, the output voltage is regulated to a fixed value acceptable for the load. In embodiments, the voltage of the treatment signal can be susceptible to wide changes based on generator settings or the status of the device tip being in contact with tissue, air, or a saline bath. Because 302 and 304 are proportional circuits, all changes on the treatment signal will be converted to the input of 306, which then provides a regulated output based on a reference voltage.

It should be understood that the individual operations used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

When considering viable loads for the energy harvesting assembly, there are several factors to consider. First, the amount of harvested energy should not affect the performance of the treatment signal. Second, the generated DC power from the harvested energy should be independent from the generator settings meaning the voltage needs to stay regulated for all different power settings on the generator. Third, the generated DC power from the harvested energy should be independent from the handpiece load, meaning the voltage needs to stay regulated for all different states of the handpiece such as no contact to tissue, in contact with the tissue, different saline or blood situations, etc. Fourth, the cost of additional circuitry shall not exceed the use of a battery. Although use of a battery comes with different downsides, cost will be a large contributor for whether additional circuitry is to be practicable.

One example load for the energy harvesting assembly could be an addition of lighting for better visibility at the distal tip of the handpiece. While some conventional handpieces may include LEDs, the LEDs are battery powered, resulting in limited and inconsistent light intensity during the life of battery. In contrast, use of the existing treatment signal will provide unlimited power for low power loads, such as one or more LEDs. Additionally, use of the treatment signal eliminates the need for additional wiring and power source, such as USB connection. Additional light at the tip of electrosurgical devices can greatly enhance a user's ability to precisely use the electrosurgical device. Further, the LED can be used to provide an indication to a user (e.g., an LED indication or other visible indication). Such an indication can provide status information for the electrosurgical device or indicated data collected or received by the electrosurgical device.

The low frequency button detection signal can also be used for energy harvesting of a load. The button detection signal has the capability to power low current devices such as an LED. In embodiments utilizing both the treatment signal and the button detection signal, a continuous low level of light can be emitted from the LED and the light can intensify when the actuator of the handpiece is pressed. In embodiments, a supercapacitor can be used to prevent the LED from blinking.

A memory and a microprocessor are viable potential loads for the energy harvesting system of the present disclosure. The memory can be configured to store identifying information of the electrosurgical device to enable detection of compatibility of the electrosurgical device with a power source. Such identifying information may include, for example, a model number, a serial number, a number of operations in which the surgical instrument has been used, and/or any other type of information. In embodiments, an RFID chip can be powered by the energy harvesting system to provide similar benefits.

The electrosurgical device can incorporate low-power communication components, such as Bluetooth low energy, to report this identifying information. In some embodiments, communication circuitry can transmit data acquired by one or more other loads of the energy harvesting system, such as sensors (e.g., a temperature sensor). In embodiments, an infrared receiver can be used for two-way communications.

In some cases, conventional generators may be limited in their ability to recognize particular instrument configurations being used and to optimize control and diagnostic processes accordingly. This can make the addition of readable data circuits to electrosurgical devices less applicable from a compatibility standpoint. However, generators can gain the requisite data reading functionality with minimal to no design changes by implementing accessories supporting this functionality through existing USB connections on the generators. In other embodiments, information communicated by the electrosurgical device can be received by a separate computing device.

Embodiments of the present disclosure can be applied to electrosurgical devices that have additional functionality such as providing saline and/or suction to the treatment site. In such embodiments, the electrosurgical device can comprise conduits, ports, or passageways and be connected to a source of fluid and/or pump. Providing suction concurrently with electrical energy to tissue advantageously allows for aspiration of debris and/or tissues cut by the electrodes. Additional actuators may be included on the handpiece to control a flow of the fluid or suction.

As previously indicated, sensors can be supported by the energy harvesting system. Sensors can include one or more of, a proximity sensor, a temperature sensor, a moisture sensor, or other sensors. These sensors can be used to activate saline or suction capabilities of an electrosurgical device.

In embodiments, electrosurgical devices that include a battery, the energy harvesting system can charge the battery. In some embodiments this can enable the battery to power additional accessories than would otherwise be feasible.

Other loads that can be supported by the energy harvesting system include a timer which can be used to improve patient safety by indicating when contact has been made with tissue for a prolonged period, a camera to improve navigation or provide a record of use for the electrosurgical device, and a speaker for providing audio cues related to patient or device status.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An electrosurgical device configured to harvest radio frequency (RF) energy, the electrosurgical device comprising:
a distal portion including two electrodes;
a proximal portion coupled to an electrical connector configured to provide a treatment RF signal and a continuous RF signal to an energy harvesting assembly housed within the electrosurgical device, the energy harvesting assembly including:
a transformer configured to isolate and reduce the voltage of the treatment RF signal;
an AC-DC converter; and
a DC-DC regulator configured to output a fixed voltage; and
one or more loads electrically coupled to the energy harvesting assembly and powered by the fixed voltage.

2. The electrosurgical device of claim 1, wherein the one or more loads are one or more of an LED, a sensor, a timer, a microprocessor, a memory, an RFID chip, communication circuitry, and a battery.

3. The electrosurgical device of claim 1, wherein the one or more loads are configured to provide identifying information about the electrosurgical device.

4. The electrosurgical device of claim 3, wherein the identifying information comprises one or more of a model number, a serial number, and a number of operations in which the surgical instrument has been used. The electrosurgical device of claim 1, wherein the AC-DC converter is diode and capacitor circuitry.

6. The electrosurgical device of claim 1, wherein the treatment signal is between 200 KHz and 3.3 MHz.

7. The electrosurgical device of claim 1, wherein the continuous signal is between 20 KHz and 300 KHz.

8. The electrosurgical device of claim 1, wherein the power of the fixed voltage is less than 10% of the treatment power.

9. The electrosurgical device of claim 1, further comprising an actuator, wherein the treatment RF signal is only provided upon actuation of the actuator.

10. A method of powering a load of an electrosurgical device comprising:
providing a radio frequency (RF) signal to the electrosurgical device and within the electrosurgical device:
circuitry;
isolating and reducing the RF signal to a range acceptable for DC circuitry;
converting the RF signal to a DC signal;
regulating the DC signal to a fixed value; and
providing the DC signal to the load at the fixed voltage.

11. The method of claim 10, wherein the load is of an LED, a sensor, a timer, a microprocessor, a memory, an RFID chip, communication circuitry, or a battery.

12. The method of claim 10, wherein the load is configured to provide identifying information about the electrosurgical device.

13. The method of claim 12, wherein the identifying information comprises one or more of a model number, a serial number, and a number of operations in which the surgical instrument has been used.

14. The method of claim 10, wherein the RF signal is provided to the electrosurgical device between 200 KHz and 3.3 MHz.

15. The method of claim 10, wherein the RF signal is provided to the electrosurgical device between 20 KHz and 300 KHz.

16. The method of claim 10, wherein converting the RF signal is accomplished by diode and capacitor circuitry.

17. The method of claim 10, wherein reducing the RF signal is accomplished by a transformer.

18. The method of claim 10, wherein the fixed voltage is based on the acceptable tolerance of the electrosurgical device.

19. The method of claim 10, wherein the power of the fixed voltage is less than 10% of the treatment power.

20. An electrosurgical device configured to harvest radio frequency (RF) energy, the electrosurgical device comprising:
an elongated distal portion including an active electrode and a return electrode;
a proximal portion coupled to an electrical three-pin connector configured to provide a treatment RF signal and a continuous RF signal to an energy harvesting assembly housed within the electrosurgical device, the energy harvesting assembly including:
a transformer configured to isolate and reduce the voltage of the treatment signal;
an AC-DC converter;
a DC-DC regulator configured to output a fixed voltage; and
a load electrically coupled to the energy harvesting assembly and powered by the fixed voltage such that the load is isolated from voltage fluctuations in the treatment RF signal.
